(19) United States
(12) Patent Application Publication
(10) Pub. No.: US 2014/0296661 A1
(43) Pub. Date: Oct. 2, 2014

(54) SLEEP STAGE ANNOTATION SYSTEM FOR INFANTS

(71) Applicant: KONINKLIJKE PHILIPS N.V., EINDHOVEN (NL)

(72) Inventors: Petronella Hendrika Zwartkruis-Pelgrim, Nuenen (NL); Gary Nelson Garcia Molina, Madison, WI (US)

(73) Assignee: KONINKLIJKE PHILIPS N.V., EINDHOVEN (NL)

(21) Appl. No.: 14/353,689

(22) PCT Filed: Oct. 22, 2012
(86) PCT No.: PCT/IB2012/055808

§ 371 Date: Apr. 23, 2014

Related U.S. Application Data
(60) Provisional application No. 61/550,964, filed on Oct. 25, 2011.

(51) Int. Cl. A61B 5/00; A61B 5/11; A61B 5/01; A61B 8/08

(52) U.S. Cl. CPC A61B 5/4812; A61B 8/08; A61B 5/0059; A61B 5/682; A61B 5/1118; A61B 5/01
USPC 600/301; 600/590; 600/438; 600/476; 600/300

(57) ABSTRACT

The present invention is related to a system for detection and/or monitoring of sleep stages in infants. Said system comprises detection means to detect and record at least one signal related to oral sucking behavior, arranging means to arrange said detecting means in an oral position of an infant, and, optionally, analyzing means to determine, from the recorded data, the sleep stage of said infant.

### FIELD OF THE INVENTION

The invention relates to the field of sleep stage annotation.

### BACKGROUND OF THE INVENTION

Assessment of sleep during infancy presents an opportunity to study the impact of sleep on the maturation of the Central Nervous System (CNS), overall functioning, and future cognitive, psychomotor, and temperament development. In addition, user research has shown that parents have a need to be informed on their child's sleep. For example, when parents need to wake up the baby, they would like to know whether it is in deep sleep or light sleep. They would prefer to wake up the baby during light sleep, since it would result in a happier baby than waking it up from deep sleep. Also parents like to know whether there are abnormalities in the sleeping patterns of their child.

Before the age of 2 months an infant's sleep is classified as either active sleep (“AS”, which later develops into REM (=Rapid eye movement) sleep, quiet sleep (“QS”, which later develops into non-REM sleep) and indeterminate sleep (“IS”, where elements of both REM and non-REM are observed).

At full term (40 weeks since conception) the background pattern detectable in an electroencephalogram (EEG) during active sleep contains activity in all frequency bands. During quiet sleep these patterns are characterized by bursts of theta and delta activity (theta and delta waves, recordable brain activity with either up to 4 Hz frequency or 4-8 Hz frequency) intermingled with periods of alpha and beta activity (alpha and beta waves, recordable brain activity with either 8-13 Hz frequency or 13-30 Hz frequency). In addition, minimal body movements can be observed during quiet sleep. After 2 months of age the sleep states can be classified as REM and non-REM. Beyond 4-6 months non-REM can be further subdivided into stages 1, 2 and slow wave sleep. Slow wave sleep can typically be seen on EEGs by 4-4.5 months of age. The behavioral patterns that are typically displayed during the various sleep stages are summarized in Table 1. From this table it can be derived that active sucking disappears during deep sleep (stages 3-4). Between 6-12 months of age sleep stage 1 and 2 can be distinguished.

TABLE 1
Summary of sleep scoring criteria in infants

| Age | State | Behavior |
|---|---|---|
| 3-6 months | Stages 1-2 | Generally quiet; may show sucking, body |
| | | movements, startles, jerks or sighs |
| | Stages 3-4 | Quiet occasional sighs; active sucking |
| | | disappears |
| | REM | Twitches, jerks, sucking, smiles, vocalization, |
| | sleep | sighs, irregular respiration, eyes open for short |
| | | periods |
| 6-12 months | Stage 1 | Generally quiet; may see sucking, body |
| | | movements, startles, jerks and/or sighs |
| | Stage 2 | Generally quiet; may see sucking, startles, |
| | | jerks and/or sighs |
| | Stages 3-4 | Quiet; occasional sighs, active sucking |
| | | disappears |
| | REM | Twitches, jerks, sucking, smiles, vocalization, |
| | sleep | sighs, irregular respiration, eyes open for short |
| | | periods; infant now quieter during REM |
| | | sleep than at an earlier age |

These stages correspond to the nomenclature provided for adults by Rechtschaffen and Kahles (1968):

| | nomenclature |
|---|---|
| stage | Rechtschaffen & Kales 1968 |
| wake | — |
| Stages 1-2 | S1/S2 |
| Stages 3-4 | S3/S4 |
| REM | |

Current methods of sleep assessment in infants include polysomnography (PSG), actigraphy, direct observations, video recordings, and pressure sensitive pads. Although PSG is the gold-standard for assessing sleep, the disadvantage of PSG is that it is a very obtrusive method, which is difficult to conduct in a home setting for prolonged periods of time. Actigraphy shows high agreement with PSG recordings and can be used for measuring the child's sleep for prolonged periods in a natural setting. The disadvantage is that it is susceptible to artifacts that result in movement despite the occurrence of sleep or the lack of movement despite waking Direct observations, video recordings and pressure sensitive pads are non-intrusive, but require the child to lay on a predefined location, such as the crib. Sleep onset and awakenings during the night are quite reliably detected using actigraphy, direct observations, video recordings, and pressure sensitive pads. However, distinguishing light and deep sleep is still challenging because the behavioral patterns that are measured by these methods are quite similar (except for sucking, see table 1).

### SUMMARY OF THE INVENTION

It is an object of the present invention to provide a sleep stage annotation system for infants which overcomes disadvantages, or shortcomings, of devices known from the prior art. It is another object of the present invention to provide a sleep stage annotation system for infants which is suitable for home use. It is yet another object of the present invention to provide a sleep stage annotation system which has good signal quality, high flexibility and high user comfort. These objects are achieved by a system and/or by a method according to the independent claims.

### BRIEF DESCRIPTION OF THE DRAWINGS

These and other aspects of the invention will be apparent from and elucidated with reference to the embodiments described hereinafter.

In the drawings:

FIG. 1 a shows a signal captured from the accelerometer mounted on the pacifier, while FIG. 1b shows an enlarged stretch of active sucking and FIG. 1c shows an enlarged stretch of passive sucking,

FIG. 2 shows the power spectrum density of the signals corresponding to active and passive sucking,

FIG. 3 shows a potential position of at least one accelerometer in a pacifier according to the invention, and

FIG. 4 shows a potential position of at least one proximity sensor or pressure sensor in a pacifier according to the invention.

### DETAILED DESCRIPTION OF EMBODIMENTS

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

According to the present invention, a system for detection and/or monitoring of sleep stages in infants is provided. Said system comprises detection means to detect and record at least one signal related to oral sucking behavior, arranging means to arrange said detecting means in an oral position of an infant, and, optionally, analyzing means to determine, from the recorded data, the sleep stage of said infant.

The inventors have surprisingly found that measuring a baby's sucking behavior provides an opportunity to distinguish between different sleep stages. In contrast to the methods from the prior art, measuring sucking behavior is a feasible and unobtrusive way of data collection, which does not affect sleeping quality of babies. Further, sucking behavior can be measured with little technical effort, which makes the system according to the invention relatively simple and affordable, and thus suitable for home use.

In a preferred embodiment according to the present invention, it is provided that said detection means is at least one sensor selected from the group consisting of:
pressure sensor,
accelerometer,
motion sensor,
muscle activity sensor,
ultrasound sensor,
proximity sensor, and/or
optical sensor.

According to Eishima (1991) an analysis of the sucking behavior of infants showed that the sucking movements consist mainly of a peristaltic tongue movement and two kinds of negative pressure. The peristaltic tongue movements are synchronized with the jaw movements. The physiological signals related to oral sucking behavior are thus jaw movements, tongue movements and pressure changes. All these signals can be determined by either of the above discussed sensors.

A pressure sensor can detect pressure changes in the oral cavity caused by sucking movements executed, among others, by the cheeks and the tongue. Peak vacuum occurs usually when the tongue is in the lowest position, and can be as high as −150±60 mmHg (Geddes et al., 2008). An accelerometer and/or a motion sensor can detect motions caused by sucking behavior. A muscle activity sensor can record muscle activity by means of suitable electrodes, e.g. in terms of an electromyogram (EMG) related to sucking behavior. An ultrasound sensor can be used to generate scans of the oral cavity, which provide information about the movements related to sucking behavior. A proximity sensor can determine changes in the distance between sensor and, e.g., the tongue or the cheeks, over time, which are caused by oral movements related to sucking behavior. An optical sensor can detect light changes caused by sucking behavior, e.g. changes in reflectance of light emitted by an infrared light source, which changes are caused by oral movements related to sucking behavior.

The discussed sensor types are well known to the skilled person. Standard sensors are available at the respective retailers and can easily be mounted into a system according to the present invention. However, the skilled person may choose other detection means to detect and record at least one signal related to oral sucking behavior without being inventive. These embodiments shall also be encompassed by the scope of the present invention, as they fully fall under the gist of the present invention.

In another preferred embodiment according to the present invention, said arranging means comprises a pacifier. A pacifier (also known as “dummy” or “soother” in some countries) is a rubber, plastic, or silicone nipple given to an infant or other young child to suck upon. In its standard appearance it has a teat, a mouth shield, and a handle. The mouth shield and/or the handle is large enough to avoid the danger of the child choking on it or swallowing it. However, in the context of the present invention the pacifier can adapt other shapes, too. Many infants use pacifiers during sleep. For example, a recent Canadian trial reports that up to 84% of infants use one (Kramer et al., 2001). The use of a pacifier is a commonly recommended practice and has been associated with a reduction in the risk for SIDS with 61% (Hauck et al., 2005).

In yet another preferred embodiment according to the present invention, said system further comprises data storage means. This is particular beneficial for infant sleep monitoring, e.g. when reasons for sleep disorders have to be detected. The device according to the invention may thus be used as a sleep data logger, which can be read out by a physician after a couple of nights to get an impression of the infant's sleep rhythm and sleep behavior. Suitable data storage means are know from the state of the art and comprise, e.g., Flash devices.

In yet another preferred embodiment according to the present invention, said system further comprises sleep stage indication means. In this embodiment, parents or a physician can control the infant's sleep stage in real time, without disturbing the infant. Preferred embodiments comprise a lighting device which has different color codes (e.g., green for light sleep and red for deep sleep), a blinking light which has different blinking frequencies for different sleep stages, or a digital display suitable for displaying numbers or letters indicating the different sleep stages. However, the skilled person may choose other sleep stage indication means without being inventive. These embodiments shall also be encompassed by the scope of the present invention.

In another preferred embodiment according to the present invention, said system further comprises means to detect whether the device is in an oral position or not. This can be done, e.g., by conductivity measurement, because, in an oral position, the system will be wetted by the infant's saliva, thus leading to increased conduction in the medium surrounding the system. Conductivity measurements can be performed by relatively simple electronic circuits which can be easily integrated into the claimed system. Another possibility is to integrate a proximity sensor of the type used in modern cell phones, where such sensor switches of the screen of the cell phone when the latter is close to the user's ear. However, the skilled person may choose other methods or sensors to detect whether the device is in an oral position or not without being inventive. These embodiments shall also be encompassed by the scope of the present invention. In all cases, a user signal can be provided in case the system is not in an oral position, e.g., because it has dropped out of the infant's mouth.

In another preferred embodiment according to the present invention, said system further comprises means to determine, from the recorded data, at least one feature selected from the group consisting of:
level of physical activity,
degree of “suction desire”, and/or
degree of appetite and/or hunger.

The level of physical activity can for example be derived from data provided by an accelerometer. The degree of “suction desire” can for example be determined by comparing actually recorded suction behavior data with comparative data stored in data storage. Further, studies have shown that infant appetite may influence sucking parameters (Geddes et al., 2008). The degree of appetite and/or hunger can thus as well be determined by the analysis of sucking behavior.

In yet another preferred embodiment according to the present invention, said system further comprises at least one device selected from the group consisting of
rechargeable battery, and/or
data communication means.

Said data communication means comprise, preferably, wireless transmitting means, e.g., under the Bluetooth standard or the WiFi standard, or as infrared light transmission, e.g., under the IrDa standard or as commonly implemented into television remote controls and similar devices. Other wireless transmission standards can however be used as well. Cable-bound data communication means comprise USB and other standard devices.

In a particularly preferred embodiment according to the invention it is provided that the system further comprises at least one device selected from the group consisting of:
actigraph
polysomnograph
temperature sensor
infrared video camera system
night vision based video camera system, and/or
pressure pads or accelerometers for sleep position sensing.

Actigraphy is a non-invasive method of monitoring human rest/activity cycles. A small actigraph unit, also called an actimetry sensor, is worn by a patient to measure gross motor activity. Motor activity often under test is that of the wrist, measured by an actigraph in a wrist-watch-like package. The unit continually records the movements it undergoes. The data can be later read to a computer and analyzed offline. In some applications, the data is transmitted and analyzed on the fly.

Polysomnography is a comprehensive recording of the biophysiological changes that occur during sleep. It is usually performed at night, when most people sleep, though some labs can accommodate shift workers and people with circadian rhythm sleep disorders and do the test at other times of day. The PSG monitors many body functions including brain (EEG), eye movements (EOG), muscle activity or skeletal muscle activation (EMG) and heart rhythm (ECG) during sleep. Optionally, breathing functions like respiratory airflow and respiratory effort indicators can as well be used, as well as peripheral pulse oximetry.

A temperature sensor can be sued to monitor the overall physiological status of the infant, because body temperature undergoes a circadian rhythm and also changes in case the infant has an infection, or other health-related issues.

An infrared video camera system and /or a night vision based video camera system can be used to detect the infant's body position or to monitor the overall level of activity. Same is applicable for pressure pads or accelerometers for sleep position sensing.

According to another aspect of the present invention, the use of a system as described above is provided for at least one purpose selected from the group consisting of
detecting sleep stages in infants in a home setting or during travelling
detecting sleep stages in child daycare
clinical child care
neonatal care
intensive child care, and/or
monitoring infant coma patients.

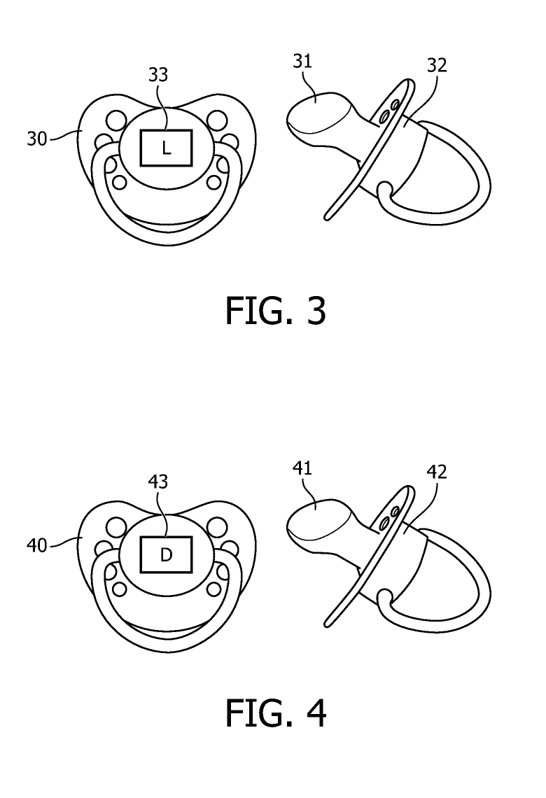

According to another aspect of the present invention, a pacifier for use with infants is provided, which pacifier comprises a system for detection and/or monitoring of sleep stages in infants system according to any of the aforementioned claims. FIG. 3 shows a pacifier 30 according to the invention, said pacifier having a teat 31 which comprises an accelerometer 32 placed inside to avoid direct contact with the baby. Rhythmic movements caused by the sucking behavior (jaw and tongue movements) can be detected to derive active sucking behavior, and stored on a data storage (not shown). In addition, a small digital display 33 can indicate the actual sleeping stage. After usage the pacifier can be connected to a PC (not shown) to download the recorded information in order to present a longitudinal overview of a child's sleeping behavior. The pacifier is equipped with a waterproof and heat resistant cover, since it needs to be cleaned and sterilized regularly.

FIG. 4 shows an alternative pacifier 40 having a teat 41, which detects sucking behavior by using a pressure sensor 42 and/or a proximity sensor. The proximity sensor is used to detect whether the pacifier is in the mouth of the infant and the pressure sensor 42 is used to detect whether active sucking takes place. In this case, a rhythmic pressure that is executed on the pressure sensors and which is related to the sucking process is evaluated. The pressure and proximity sensors are placed on the upper part of the teat. In addition, a small digital display 43 can indicate the actual sleeping stage.

### EXPERIMENT DESCRIPTION

A 3D accelerometer (same type as used in the DirectLife Activity Monitor marketed by Philips) was mounted on a pacifier as illustrated in FIG. 3. The accelerometer can sample acceleration at 100 Hz and was equipped with an internal data logger which could be read out by a computer. A participant was asked to use the pacifier for 30 seconds and produce two types of sucking behavior, active and passive, in which “active sucking” means that sucking movements are actively carried out and “passive sucking” refers to the fact of just holding the pacifier in the mouth. Events were used to annotate the sucking behavior.

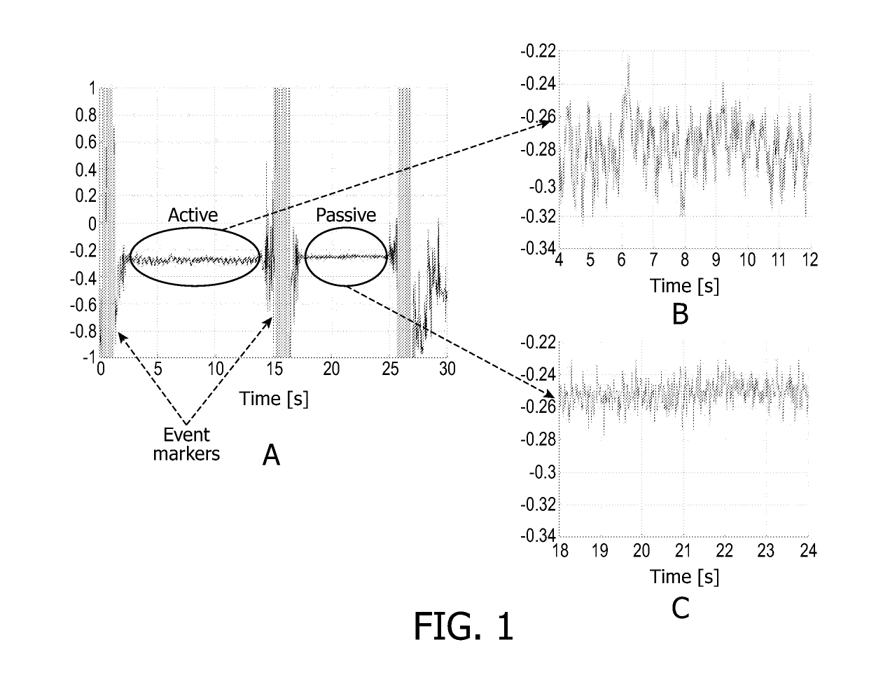

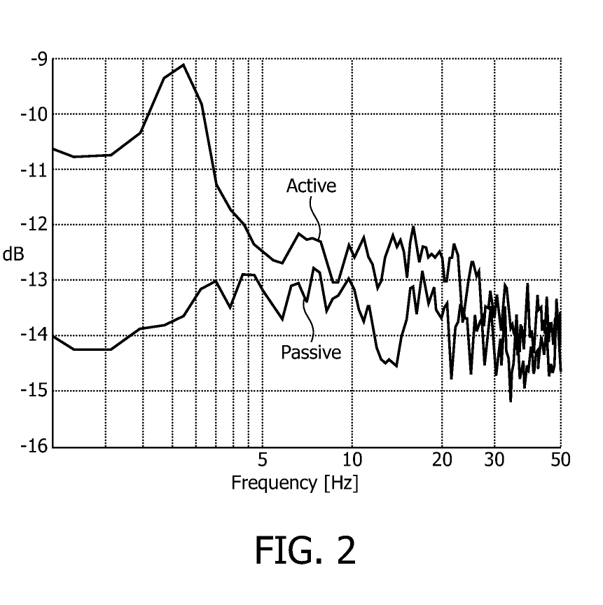

The rhythmic pattern in the active sucking can be seen in FIG. 1b. The spectral analysis of the signals from the active and passive sucking period is reported in FIG. 2. The rhythmic nature of the active sucking can be clearly seen as a peak at around 2.7 Hz in the spectral representation of the active sucking in FIG. 2.

Simple thresholding in the frequency domain can be used to detect the presence of rhythmic patterns in the sucking activity and from there derive the infant's sleep behavior.

### REFERENCES

Eishima K. (1991). The analysis of sucking behavior in newborn infants. Early Hum Dev. 1991 Dec;27(3):163-73.

Kramer, M. S., Barr, R. G., Dagenais, S., Yang, H., Jones, P., Ciofani, L., & Jane, F. (2001). Pacifier use, early weaning, and cry/fuss behavior: a randomized controlled trial. Journal of the American Medical Association, 286, 322-326.

Hauck, F. R., Omojokun, O. O., Siadaty. M. S. (2005). Do pacifiers reduce the risk of sudden infant death syndrome? A meta-analysis. Pediatrics, 116, e716-e723

Insana, S. P., Gozal, D., & Montgomery-Downs, H. E. (2010). Invalidity of one actigraphy brand for identifying sleep and wake among infants. Sleep Medicine, 11, 191-196.

Rechtschaffen A. and Kales A, “A manual of standardized terminology, techniques and scoring system for sleep stages of human subjects,” U.S. National Institute of Neurological Diseases and Blindness, Neurological Information Network, Bethesda, Md., 1968.

Geddes D T, Kent J C, Mitoulas L R, Hartmann P E (2008): Tongue movement and intra-oral vacuum in breastfeeding infants. Early Human Development (2008) 84, 471-477

1. A system for detection and/or monitoring of sleep stages in infants, said system comprising detection means to detect and record at least one signal related to oral sucking behavior, and arranging means to arrange said detection means in an oral position of an infant, wherein said detection means is at least one sensor selected from the group consisting of:
pressure sensor,
accelerometer,
motion sensor,
muscle activity sensor,
proximity sensor,
ultrasound sensor, and/or
optical sensor.

2. The system according to claim 1, wherein
analyzing means to determine, from the recorded data, the sleep stage of said infant.

3. The system according to claim 1, wherein said arranging means comprises a pacifier.

4. The system according to claim 1, wherein said system further comprises data storage means.

5. The system according to claim 1, wherein said system further comprises sleep stage indication means.

6. The system according to claim 1, wherein said system further comprises means to detect whether the device is in an oral position or not.

7. The system according to claim 1, wherein said system further comprises means to determine, from the recorded data, at least one feature selected from the group consisting of:
level of physical activity,
degree of “suction need”, and/or
degree of appetite and/or hunger.

8. The system according to claim 1, wherein said system further comprises at least one device selected from the group consisting of
rechargeable battery, and/or
data communication means.

9. The system according to claim 1, wherein said system further comprises at least one device selected from the group consisting of
actigraph
polysomnograph
infrared video camera system
temperature sensor
night vision based video camera system, and/or
pressure pads or accelerometers for sleep position sensing.

10. Use of a system according to claim 1, wherein at least one purpose selected from the group consisting of:
detecting sleep stages in infants in a home setting or during travelling
detecting sleep stages in child daycare
clinical child care
neonatal care
intensive child care, and/or
monitoring infant coma patients.

11. A pacifier for use with infants, said pacifier comprising a system for detection and/or monitoring of sleep stages in infants system.
